(19) United States
(12) Patent Application Publication
(10) Pub. No.: US 2017/0074872 A1
(43) Pub. Date: Mar. 16, 2017

(54) HIGH-THROUGHPUT SINGLE CELL SORTING USING MICROBUBBLE WELL ARRAYS

(71) Applicant: Nidus Biosciences, LLC, Rochester, NY (US)

(72) Inventors: Lisa A. DeLouise, Rochester, NY (US); James J. Kobie, Rochester, NY (US)

(73) Assignee: Nidus Biosciences, LLC, Rochester, NY (US)

(21) Appl. No.: 15/209,361

(22) Filed: Jul. 13, 2016

Related U.S. Application Data
(60) Provisional application No. 62/191,666, filed on Jul. 13, 2015.

(51) Int. Cl. G01N 33/543; G01N 33/68

(52) U.S. Cl. CPC G01N 33/54366; G01N 33/6854; G01N 33/54346

(57) ABSTRACT

The present invention provides a microfabricated device and methods for high throughput single cell screening of a heterogeneous population. The present invention is partly based upon but not limited to sorting by monitoring cell secreted factors that accumulate in time (hours, days, weeks) as cells are cultured in the microbubble well niche the architecture of which facilitate the accumulation. In certain embodiments, the device and method comprises a means to identify effective drugs for personalize therapeutics such as but not limited to discovery of monoclonal antibody therapeutics.

### CROSS REFERENCE TO RELATED APPLICATION

This application claims priority to Provisional U.S. Application 62/191,666, filed Jul. 13, 2015, which is hereby incorporated by reference in its entirety.

### STATEMENT REGARDING FEDERALLY SPONSORED RESEARCH OR DEVELOPMENT

This invention was made with government support under The University of Rochester Clinical and Translational Science Institute that is supported in part by grants UL1 TR000042, KL2 TR000095, and TL1 TR000096 from the National Center for Advancing Translational Sciences (NCATS), a component of the National Institutes of Health (NIH). The government has certain rights in the invention.

### FIELD OF THE INVENTION

The present invention provides a microfabricated device and methods for high throughput single cell screening of a heterogeneous population. The present invention is partly based upon but not limited to sorting by monitoring cell secreted factors that accumulate in time (hours, days, weeks) as cells are cultured in the microbubble well niche the architecture of which facilitate the accumulation. In certain embodiments, the device and method comprises a means identify effective drugs for personalize therapeutics such as but not limited to discovery of monoclonal antibody therapeutics.

### BACKGROUND OF THE INVENTION

The ability to sort cells from heterogeneous population and to study them at the single cell level provides unique opportunities for drug discovery, identification of tumor initiating cancer stem cells and for understanding signaling pathways in disease [1-3]. This capability is particularly advantageous for the production of monoclonal antibodies, which requires the sorting of potentially rare (1 in >10⁴) antibody producing cells from a heterogeneous population. Monoclonal antibodies (mAb) are a rapidly growing class of human therapeutics with a market size of roughly $78 billion in 2012 [4]. Their ability to specifically recognize and bind antigens of interest with high affinity holds vast potential as treatments for diseases ranging from autoimmune disorders to infectious diseases and cancer therapeutics [5-7].

Microfabricated technologies have become increasingly popular in cell biology and disease state research due to their ability to capture and monitor single cells in physiologically relevant microenvironments [8-13]. Within these in vitro microenvironments, heterogeneous cell populations can be sorted and independently interrogated within one device that overcomes many limitations of standard cell culture assay systems [14, 15]. For example, use of the 96-well plate format imposes the constraint of a high media volume to surface area ratio [16], which hinders cell self-conditioning of wells when seeded under limiting dilution conditions [17]. Relatively large reagent volumes, long processing times, and the necessity to use many plates to assay for minority cell types or secreted soluble factors (e.g. cytokines, antibodies) are additional limitations that can be overcome using microfabricated systems [14, 18, 19]. The attributes of a low cell culture volume, customizable surface chemistry, and the ability to fabricate high density micro-well arrays, are particularly advantageous for immune system research in which both single cell studies and interactions between B and T cells can be specifically probed and their secreted products (cytokines and antibodies) can be quantified [14, 20-22].

The use of microfabricated devices to quantify immune cell secreted products is particularly useful in the field of monoclonal antibody discover [2, 14, 23-26]. Conventional mAb production involves fusing splenocytes from immunized mice with an immortalized myeloma cell line. The resulting hybridoma cells are cultured under limiting dilution conditions (<1 cell per well) in microtiter plates for 7 to 14 days to allow for clonal expansion. The culture supernatants are then tested for antigen specificity using Enzyme Linked Immunosorbent Assay (ELISA) methods to identify the wells containing cells of interest [22, 23]. While this method is effective, the process is laborious, time consuming and costly. Moreover, relatively few (˜10³) of the hybridoma cells produced can be tested and therefore potentially high affinity mAbs may be missed.

To expand and simplify the antibody discovery process microfabrication technologies have been exploited to develop novel single cell high-throughput methods for screening >10⁵ cells from hybridoma cells or screening antigen specific B cells from human peripheral blood. There are several single cell methods reported for detecting antibody secreting cells (ASC) including antigen arrays [24], droplet based fluidic systems [2], and micro-well techniques including Microengraving [14, 25] and ISAAC [26]. Microengraving utilizes large arrays of shallow cuboidal micron scale pits formed in polydimethylsioxane (PDMS) to seed cells. The array is capped with a glass slide functionalized to bind secreted mAbs. After ˜2-4 hours in culture the slide is removed from the array, treated with a secondary reporter and then used as a template to locate positive wells containing the cell(s) producing the mAb of interest [14]. The ISSAC technique similarly uses shallow micro-well arrays formed in PDMS to seed cells, however mAb detection is done through direct binding of cell secretions to an antigen specific surface coating [26]. Direct detection of fluorescence around the exterior of a well greatly simplifies the process of locating positive wells. While the aforementioned techniques make vast improvements over the conventional ELISA cell screening process, they still suffer from various drawbacks. In Microengraving, the array capping process limits the nutrient exchange within the pits and thus limits the time allowed for detecting mAb secretions to only a few hours and therefore only ASC that secrete at a high rate can be detected. While the IS SAC technique does not rely on a cap for signal generation, the open well architecture allows for the loss of cell secretions over time by diffusion and dilution into the bulk media. In shallow well architectures the cells may be easily dislodged by turbulent fluid flow, creating uncertainty in being able to recover the specific cell of interest. Neither system allow for clonal expansion of cells which could greatly increase detection sensitivity and thus enable the discovery of potentially high affinity mAbs that are secreted at a low rate.

To overcome these limitations, we have developed a microfabricated device platform based on microbubble well array technology for culturing single cells for hours to days to weeks where they can be sorted based on clonal proliferation, clonal morphology, cell secreted factors, cell and cell-cell function and other means. MB wells are deep (80-250 μm) spherical compartments with 40-100 μm diameter circular openings fabricated in PDMS using the gas expansion molding process [18, 27]. We have shown that the unique MB well architecture facilitates the accumulation of cell secreted factors while allowing for sufficient nutrient and waste exchange to enable cell proliferation [28]. Although having similar goals to Microengraving and ISSAC, the MB well architecture allows for a novel means to sort single and multiple cells that is not possible with shallow well device architectures. The high aspect ratio and spherical architecture of the microbubble well provides a niche for cells to proliferate and their secreted factors to concentrate over hours to days to weeks. Capping the array to prevent loss of secreted factors is not needed and thus simplifies the detection and recovery of cells from wells of interest. Previous studies have shown MB well arrays are an optimum device for culturing various cell lines over time periods ranging from days to weeks with intermittent media changes without concern for dislodging cells from the wells [29-32]. The spherical geometry of the MB well allows for the reduction in shear stresses at the base of the MB both aiding in the growth and propagation of the cells as well as reducing the need for a capping process for cell and secreted factor containment [30]. Cells in MB wells are not easily displaced by fluid flow and in fact it takes several hours for nonadherent cells to exit the wells of inverted chips [29]. Moreover, due to the material properties of the chip we can take advantage of surface tension to apply coatings to the chip surface that differs from the coating that maybe deposited into the MB well itself.

Here, we disclose the use of the microbubble well device for conducting high-throughput single cell screening assays to identify and recover specific cells and cell colonies that grow in a MB well over time by monitoring cell secretions that accumulate in the MB well due to its unique architecture (shape, size, dimensions). When the concentration of the secreted products rise to a sufficient level and binding to a multivalent reporter detection signal is observed. This invention encompasses the discovery of monoclonal, synthetic antibodies, and the like. One skilled in the art will understand that based upon the disclosure provided herein, the crucial feature of the invention is that the antibody binds specifically with a reporter (antigen, cytokine, protein, peptide, nanoparticle etc.) of interest. That is, the antibody of the invention recognizes a reporter construct of interest including an antigen or antibody or a fragment thereof (e.g., an immunogenic portion or antigenic determinant thereof), causing immunoprecipitates to form in the unique microenvironment of the MB well. One skilled in the art will understand that the immunoprecipitation signal can be converted to a more homogeneous signal using affinity capture coatings and fluorescently labeled reporters, antigen or antigen coated fluorescent beads, which are standard methods well-known in the art but applying these coatings selectively in the MB well while the invention disclosed a process to apply a coating to block cell and/or protein binding on the chip surface.

### SUMMARY OF THE INVENTION

The present invention provides a microfabricated device and methods for high throughput single cell screening of a heterogeneous population. The present invention is partly based upon but not limited to sorting by monitoring cell secreted factors that accumulate in time (hours, days, weeks) as cells are cultured in the microbubble well niche the architecture of which facilitate the accumulation. In certain embodiments, the device and method comprises a means identify effective drugs for personalize therapeutics such as but not limited to discovery of monoclonal antibody therapeutics.

Note that in the following embodiments, embodiment 1 is explicitly recognized as including other sorting methods, e.g. clonal proliferation, clonal morphology, drug resistance, cell adhesion, secretion rate, surface markers, and cell functional characteristics including but not limited to the ability to block or promote signaling pathways, or to enhance opsonization to name a few.

In embodiment 1, the present invention is directed to a method using a microfabricated device for cell culture, sorting and analysis in situ or ex situ, where the microfabricated device comprises one or more curvilinear microbubble well cavities embedded in a non-glass substrate, where the opening into the cavity is smaller in diameter than the diameter of the cavity is at its largest extent so as to create a microenvironmental niche into which one or more cells can be seeded and cultured for a period of time of hours to days to weeks so that their secreted product(s) can accumulate to promote cell survival and/or proliferation.

In embodiment 2, the present invention is directed to a method using a microfabricated device in embodiment 1 with cavity openings of 20-200 microns in diameter.

In embodiment 3, the present invention is directed to a method using a microfabricated device in embodiment 1 with cavity openings of 40 to 60 microns in diameter.

In a embodiment 4, the present invention is directed to a method using a microfabricated device in embodiment 1 with circular, triangular, rectangular, or square cavity openings.

In embodiment 5, the present invention is directed to a method using a microfabricated device in embodiment 1 with a maximum cavity diameter that is larger than the cavity opening.

In embodiment 6, the present invention is directed to a method using a microfabricated device in embodiment 1 with a maximum cavity diameter that is about 2 to 4 times larger than the cavity opening.

In embodiment 7, the present invention is directed to a method using a microfabricated device in embodiment 1 comprised of one or more cavities in an array.

In embodiment 8, the present invention is directed to a method using a microfabricated device in embodiment 1 with an array of cavities from 2 to 1 million or more.

In embodiment 9, the present invention is directed to a method using a microfabricated device in embodiment 1 comprising a substrate material with an elastic modulus similar to in vivo tissue microenvironment.

In embodiment 10, the present invention is directed to a method using a microfabricated device in embodiment 1 comprising a substrate material with an elastic modulus similar to in vivo soft tissue microenvironment.

In embodiment 11, the present invention is directed to a method using a microfabricated device in embodiment 1 where the substrate material has an elastic modulus in the range of 100 to 1000 KPa.

In embodiment 12, the present invention is directed to a method using a microfabricated device in embodiment 1 that is comprised of a polymer substrate material with an elastic modulus in the range of 100 to 1000 KPa.

In embodiment 13, the present invention is directed to a method using a microfabricated device in embodiment 1 where the substrate material is a clear polymer material with elastic modulus in the range of 100 to 1000 KPa such as polydimethylsiloxane (PDMS).

In embodiment 14, the present invention is directed to a method using a microfabricated device in embodiment 1 into which cells are seeded.

In embodiment 15, the present invention is directed to a method using a microfabricated device in embodiment 14 where the cells are selected from the group consisting of mouse hybridoma cells, CHO cells, or B cells derived from human or animal peripheral blood or lymphoid organs.

In embodiment 16, the present invention is directed to a method using a microfabricated device in embodiment 1 into which the number of cells seeded per cavity is controlled.

In embodiment 17, the present invention is directed to the method of claim 16, where the number of cells seeded per cavity follows a statistical population.

In embodiment 18, the present invention is directed to the method of claim 17, where the number of cells seeded per cavity follows a statistical population defined by Poisson distribution.

In embodiment 19, the present invention is directed to a method using a microfabricated device in embodiment 1 into which the number of cells seeded per cavity follows a statistical distribution with preferred seeding of ˜37% of the cavities have 0 cells, ˜37% have 1 cell, ˜18% have 2 cells and ˜8% have 3 cells.

In embodiment 20, the present invention is directed to a method using a microfabricated device in embodiment 6 that provides a microenvironmental niche for a single cell or multiple cells seeded into a cavity to readily condition with autocrine or paracrine secreted factors to support cell survival and clonal or colony proliferation.

In embodiment 21, the present invention is directed to a method using a microfabricated device in claim 14 further configured to permit visual inspection of cells in cavities after seeding and over time to quantify cell proliferation so as to distinguish cells that die from cells that undergo rapid proliferation.

In embodiment 22, the present invention is directed to a method using a microfabricated device in embodiment 14 into which the seeded cells are cultured for a period of time ranging from hours to several days in media that contains supplements and reporters.

In embodiment 23, the present invention is directed to a method using a microfabricated device in embodiment 22 in which the reporter is a fluorescently or chromogenic tagged antigen, peptide, cytokine, antibody or other protein or nanoparticle that will bind to cell secreted factors.

In embodiment 24, the present invention is directed to a method using a microfabricated device in embodiment 14 in which the reporter binds to cell secreted factors causing a precipitation reaction.

In embodiment 25, the present invention is directed to a method using a microfabricated device in embodiment 1 in which the cavities are coated with a functional biomolecule and the device surface is coated separately with a cell and/or protein-blocking reagent.

In embodiment 26, the present invention is directed to a method using a microfabricated device in embodiment 25 where an unprimed chip having air in the microbubble wells is exposed to a high surface tension liquid containing a coating protein.

In embodiment 27, the present invention is directed to a method using a microfabricated device in embodiment 26 where the liquid has a surface tension >40 dyne-cm, and preferably 70 dyne-cm.

In embodiment 28, the present invention is directed to a method using a microfabricated device in embodiment 26 where the coating protein is a blocking agent such as bovine serum albumin, casein, polyethyleneglycol (PEG) or other blocking agents know in the field.

In embodiment 29, the present invention is directed to a method using a microfabricated device in embodiment 26 where the coating protein is allowed to react with the chip surface for a period of time from 1 to 24 hours at room temperature (RT) or at 4 C, preferably 2 hrs at RT.

In embodiment 30, the present invention is directed to method using a microfabricated device in embodiment 26 where the coating protein is allowed to react with the chip surface for a period of time from 1 to 24 hours at room temperature (RT) or at 4 C, preferably 2 hrs at RT.

In embodiment 31, the present invention is directed to a method using a microfabricated device in embodiment 26 where the coating protein is washed off and replace with a buffer.

In embodiment 32, the present invention is directed to a method using a microfabricated device in embodiment 31 where the chip immersed in buffer is placed in a vacuum to draw the buffer into the microbubble wells to attain a primed array.

In embodiment 33, the present invention is directed to a method using a microfabricated device in embodiment 32 in which the primed array is placed in a second solution containing a bioactive molecule that will coat the inside of the microbubble cavity.

In embodiment 34, the present invention is directed to a method using a microfabricated device in claim 33 where the bioactive molecule is allowed to react with the microbubble well surface for a period of time from 1 to 24 hours at room temperature (RT) or at 4 C, preferably 2 hrs at RT.

In embodiment 35, the present invention is directed to a method using a microfabricated device in claim 33 in which the bioactive molecule can affect cell function such as an extracellular matrix protein or capture cells or cell secreted products on the cavity surface.

In embodiment 36, the present invention is directed to a method using a microfabricated device in claim 25 in which the cell secreted products that are captured on the cavity surface are detected with a fluorescently or chromogenic tagged antigen, peptide, cytokine, antibody or other protein or nanoparticle reporter.

In embodiment 37, the present invention is directed to a method using a microfabricated device in claim 1 or 25 in which the cell secreted products that precipitate or are captured on the cavity surface are detected with a reporter for which the signal strength in individual cavities is monitored over time to identify cavities containing cells that secrete product early and at a fast rate.

In embodiment 38, the present invention is directed to a method using a microfabricated device in claim 1 or 25 into which two or more distinct cells types are seeded into the microcavity niche to observe functional readouts.

In embodiment 39, the present invention is directed to a method using a microfabricated device in claim 14 or 25 into which cells are seeded in micro-niche cavities and cultured in time (hours to days to weeks) to observe clonal proliferation and morphology.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic showing construction of monovalent antigen coated onto a nanoparticle to produce a multiantigen display for immunoprecipitation to occur.

FIG. 2 is a schematic of the coating process used to capture IgG secretions from the SA13 cells onto the surface of the MB and to detect them in an antigen specific manner. A) Unlabeled α-IgG coated on inner MB. B) Cells seeded, producing IgG that binds coating. C) Fluorescently labeled tetanus toxoid binds IgG layer.

FIG. 3 shows an example of a region (A) within a MB well array in bright field and fluorescence were SA13 hybridoma cells were seeded and cultured for 6 days in media that contained FITC-anti-IgG antibody. It is clear from the fluorescence images that the wells containing cells that secreted antibody light up and show evidence of speckle due to immunoprecipitation reaction. In control arrays (B) where no cells were seeded there is no evidence for immunoprecipitation.

FIG. 4 shows a magnified view of wells from FIG. 3 (Row A, Column ii) exemplifying a strong positive immunoprecipitation signal, presumed negative and weak positive wells.

FIG. 5 show detection of antigen specific IgG using multivalent antigen coated nanoparticles, A) SA13 cells with unfunctionalized PS-NP, B) SA13 cells with tetanus toxoid functionalized PS-NP.

FIG. 6 shows fluorescence images of PS-NPs functionalized with tetanus toxoid and culture with in MB arrays containing ARH-77 cells (A&B) and SA13 cells (C&D) cells before and after washing. Results show more bead accumulation in MB wells with SA13 cells but there are significant concerns with NP colloidal stability.

FIG. 7 is a magnified view of SA13 incubated with toxoid coated nanoparticles post wash (FIG. 6) showing wells that retained fluorescence correspond to wells which contain high levels of SA13 cells.

FIG. 8 shows detection of cell secreted products using affinity capture coatings. Ring development signifying detection of IgG secretion from MBs of interest. A) Fluorescent image, B) Corresponding bright-field image.

FIG. 9 shows ring detection using anti-IgG affinity capture coating and IgG specific detection. Ring signal was only detected for SA17 cells that secrete IgG whereas no fluorescent signals is detected for the ARH-77 and CCL-119 cells that do not secrete IgG.

FIG. 10 shows that detection of secreted products does not scale with the number of cells per well as not all cells within a population secrete antibody.

FIG. 11 shows ring signal detection using anti-IgG affinity capture coating and fluorescently labeled antigen.

FIG. 12 shows IgG signal detection sensitivity using ring detection method.

FIG. 13 Shows primary B cell proliferation in MB well. A representative MB well was tracked over the course of 4 days to monitor the cell proliferation of primary B cells. A) Bright-field image of the MB on day 0 immediately after cell seeding well contains 2 cells. B) Bright-field image of same MB 4 days later. C) Live/dead stain of MB on day 4. Green represents live cells while red represents dead cells.

FIG. 14 shows a stitched image scan of MB array containing approximately 1000 MBs seeded with primary human B cells showing detection of secreted IgG using an anti-IgG Capture Coating 2 days post seeding. MB with fluorescent rings around them are considered positive wells.

FIG. 15 shows a zoomed in image of live human B cells secreting IgG.

FIG. 16 shows images of commercial Eppendorf (A, B) equipment used to move a capillary into a microbubble well containing cells (C) and ejecting the cells (D) from the MB well.

FIG. 17 shows PCR amplification of immunoglobin genes on cells recovered from microbubble wells.

FIG. 18 shows primers used for PCR amplification of Immunoglobin Genes.

FIG. 19 shows CnP approximations at various cell seeding concentrations; equations relating the cells seeding statistics that follow Poisson Distribution.

FIG. 20 shows Elispot data showing the % of IgG and Toxoid specific antibody secreting SA13 cells.

FIGS. 21 and 22 show the references cited in the present application.

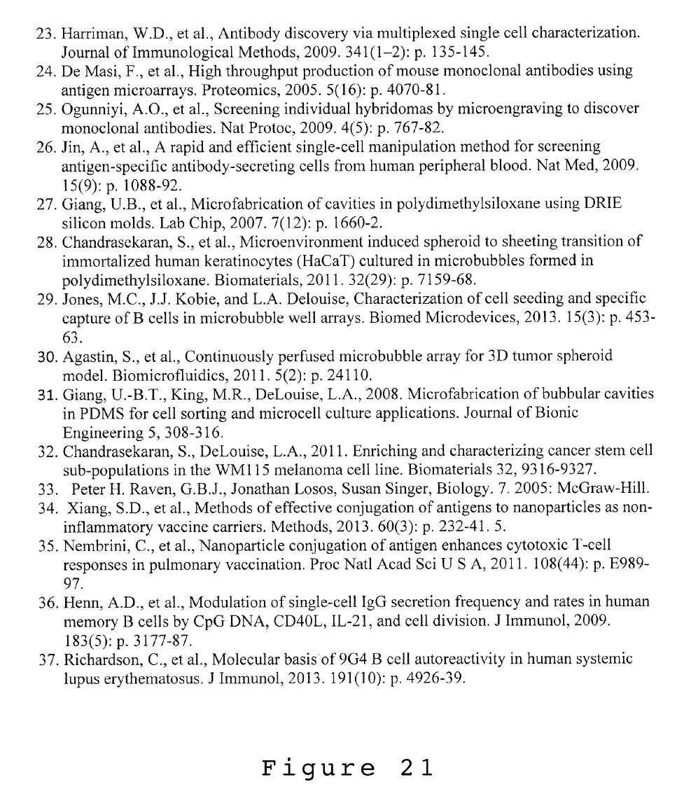

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods. Note that “about” is sometimes given using a tilde, i.e., “˜”.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. Antibodies discovered by using the present invention may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to at least one portion of an intact antibody and refers to the antigenic determining variable regions of an intact antibody. Examples of antibody fragments include, but are not limited to, Fab, Fab′, F(ab′)2, and Fv fragments, linear antibodies, sdAb (either V_{L} or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

By the term “synthetic antibody” as used herein, is meant an antibody which is generated using recombinant DNA technology, such as, for example, an antibody modified to enhance binding specificity or to humanize the antibody. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

By the term “antigen-bearing moiety” as used herein, is meant a molecule to which an antibody binds.

“Biological sample,” or simply “sample”, as that term is used herein, means a sample, such as one that is, but need not be, obtained from an animal, which sample is to be assessed for the presence of a biological organism, or component thereof, such that the sample can be used to assess the presence, absence and/or level, of an antigen, or ligand, of interest according to the methods of the invention. Such sample includes, but is not limited to, any biological fluid (e.g., blood, lymph, semen, sputum, saliva, phlegm, tears, and the like), fecal matter, a hair sample, a nail sample, a brain sample, a kidney sample, an intestinal tissue sample, a tongue tissue sample, a heart tissue sample, a mammary gland tissue sample, a lung tissue sample, an adipose tissue sample, a muscle tissue sample, and any sample obtained from an animal that can be assayed for the presence or absence of an antigen. Further, the sample can comprise an aqueous sample (e.g., a water sample) to be assessed for the presence of an organism, or a component thereof, such as a drinking water sample, before or after any treatment, wherein the presence of a biological organism (e.g., a Cryptosporidium organism) is assessed.

As used herein, the term “fragment” as applied to a nucleic acid, may ordinarily be at least about 20 nucleotides in length, preferably, at least about 30 nucleotides, more typically, from about 40 to about 50 nucleotides, preferably, at least about 50 to about 80 nucleotides, even more preferably, at least about 80 nucleotides to about 90 nucleotides, yet even more preferably, at least about 90 to about 100, even more preferably, at least about 100 nucleotides to about 150 nucleotides, yet even more preferably, at least about 150 to about 200, even more preferably, at least about 200 nucleotides to about 250 nucleotides, yet even more preferably, at least about 250 to about 300, more preferably, from about 300 to about 350 nucleotides, preferably, at least about 350 to about 360 nucleotides, and most preferably, the nucleic acid fragment will be greater than about 365 nucleotides in length.

As used herein, the term “fragment” as applied to a polypeptide, may ordinarily be at least about 20 amino acids in length, preferably, at least about 30 amino acids, more typically, from about 40 to about 50 amino acids, preferably, at least about 50 to about 80 amino acids, even more preferably, at least about 80 amino acids to about 90 amino acids, yet even more preferably, at least about 90 to about 100, even more preferably, at least about 100 amino acids to about 120 amino acids, and most preferably, the amino acid fragment will be greater than about 123 amino acids in length.

By the term “Fab/phage” as used herein, is meant a phage particle which expresses the Fab portion of an antibody.

By the term “scFv/phage” are used herein, is meant a phage particle which expresses the Fv portion of an antibody as a single chain.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein or peptide sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein, is meant an antibody, or a ligand, which recognizes and binds with a cognate binding partner present in a sample, but which antibody or ligand does not substantially recognize or bind other molecules in the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides a microfabricated device and methods for high throughput single cell screening of a heterogeneous population. The present invention is partly based upon but not limited to sorting by monitoring cell secreted factors that accumulate in time (hours, days, weeks) as cells are cultured in the microbubble well niche the architecture of which facilitate the accumulation. In certain embodiments, the device and method comprises a means identify effective drugs for personalize therapeutics such as but not limited to discovery of monoclonal antibody therapeutics.

For example, in one embodiment, the present invention provides a device and method to identify cell-secreted factors by precipitation. For example, in one embodiment, the present invention provides a device and method to identify monoclonal and polyclonal antibody secretions by immunoprecipitation. Another embodiment of the present invention provides a device and method to identify antigen specific monoclonal and polyclonal antibody secretions by immunoprecipitation. In certain embodiments, the device and method comprises a means identify antigen specific monoclonal and polyclonal antibody secretions from mouse hybridoma cells, CHO cells, or B cells derived from human or animal peripheral blood or lymphoid organs.

In one embodiment of the present invention in order for immunoprecipitation to take place the immunoglobin present must be able to bind a multivalent reporter (antigen or antibody) to form large polymerized agglutinates. Once a sufficient size threshold is reached the agglutinates settle forming a precipitate [33]. As would be understood by those skilled in the art, the polymerization of immuno-complexes requires multi-valency in the reporter and in some cases, particularly for monoclonal antibodies which target a reporter with only one epitope the creation of a multivalent reporter construct would be needed to induce immunoprecipitation. Those skilled in the art would recognize that a multivalent reporter construct can be created by displaying multiple copies of the antigen onto nanoparticle detection system.

In one embodiment, the present invention provides a device and method identify cell secreted factors by capturing them to the surface of the MB well. For example, in one embodiment, the present invention provides a device and method to identify monoclonal and polyclonal antibody secretions by capturing them to the surface of the MB well. Another embodiment of the present invention provides a device and method to identify antigen specific monoclonal and polyclonal antibody secretions by capturing them to the surface of the MB well. In certain embodiments, the device and method comprises a means identify antigen specific monoclonal and polyclonal antibody secretions from mouse hybridoma cells, or CHO cells, or B cells from human or animal peripheral blood or lymphoid organs.

The present invention provides microfabricated device and methods for conducting high throughput single cell screening. The microfabricated device is comprised of an array of curvilinear cavities fabricated in a low elastic modulus polymer such as polydimethylsiloxane which more appropriately mimics the in vivo soft tissue microenvironment compared to hard tissue culture plastic. The methods are described using this device to conduct single cell high throughput screens of heterogeneous cell samples. Single cells are sorted by but not limited to cell secreted factors.

### Microbubble Well Array Fabrication

In one embodiment, the present invention includes a screening method for single cell sorting using microbubble well arrays. The MB well arrays were formed in PDMS using the gas expansion molding (GEM) process [18, 27]. Briefly, the process utilizes a silicon wafer mold that contains an array of cylindrical pits of 60 μm in diameter and 150 μm in depth (with each feature spaced 4× apart from one another on a square lattice. These features were etched utilizing the Bosch deep reactive ion etch process (Plasma Therm 770, MEMS and Nanotechnology Exchange LLC, Reston, Va.). To cast the PDMS MB well array, a 10:1 base to elastomer curing agent ratio was used (Sylgard, Dow Corning, USA). The prepolymer was mechanically mixed in a 50 mL conical tube with a plastic pipette for approximately 20-30 seconds or until adequate mixing was achieved, as noticed by the formation of air bubbles within the suspension. The PDMS premix was then poured onto the silicon wafer to achieve a PDMS thickness of ˜2 mm. The PDMS is left to self-level at room temperature for ˜10 minutes. Following the self-leveling process the residual bubbles left in the PDMS layer are removed by mechanical puncturing with a sterile pipette tip. The silicon wafer with the PDMS premix is then moved to the 100° C. oven to cure for 1 hour. A spherical MB well forms over each deep pit in the silicon wafer. The size of the MB wells produced have circular openings of approximately 40-200 μm in diameter and overall diameter of approximately 60-300 μm but preferably 60 μm in diameter with overall diameter of 120 μm (U.S. patent application Ser. Nos. 13/469,184 and 13/304,843). Following the MB well formation process the wafer and the PDMS cast are removed from the oven and the PDMS cast is peeled away from the silicon wafer mold. The chips are then cut to the desired size using a surgical blade and stored at room temperature in an enclosed Petri dish for future experiments.

### Microbubble Array Preparation for Cell Seeding

In one embodiment, the present invention includes a single cell screening method whereby cells are seeded into the microbubble well arrays. To prepare the arrays for cell seeding the MB well array chips are placed on a glass slide with the MB well openings facing down. The slide and the arrays were then treated for 60-90 seconds in oxygen plasma to increase the backside chip wettability (March Instruments Inc., USA). Following the plasma treatment, the chips were moved into separate wells of a 24 well plate and submerged in 1 mL of 1:1 DI water and ethanol. The 24 well plate containing the MB arrays was then placed in a vacuum chamber and degassed for 2-3 minutes or until the MB array became clear, signifying successful priming (infiltration of liquid into the MB wells) of the arrays. This step also serves to sterilize the chip before cell seeding. The MB arrays were then moved to new wells of the 24 well plate and submerged in 1 mL of 1×PBS (BP13351 Fisher BioReagents, USA) under sterile conditions. The 24 well plate with the MBs was then moved to a 37° C. incubator where they were stored for a minimum of 20 hours. Following incubation in PBS chips were moved to new wells on the 24 well plate and incubated in fresh RPMI 1640 (Gibco A10491-01, Invitrogen Corp., USA) for 10-15 minutes at 37° C. to allow for priming of the MBs with media. The chips were then moved into new wells and 1 mL of cell suspension was pipette into the wells at a seeding density of 15,000 cells/cm² to achieve efficient single cell capture. Chips were incubated for approximately 10 minutes before being rinsed and placed into new wells containing fresh RPMI 1640 (Gibco A10491-01, Invitrogen Corp., USA). Previous studies quantified how the MB well array seeding statistics (% of wells with 0, 1, 2, 3 etc. cells/well) depends on the cell stock concentration, incubation time, and the MB well opening size [29,30]. The seeding statistics for the conditions used in these studies (n=3 seeding trials) produced arrays with 62%±5% of wells with 0 cells, 28%±2% of wells with 1 cells, 7%±4% of wells with 2 cells, 2%±2% of wells with 3 cells, and 0.3%±0.6% of wells with >4 cells/well.

### Assay for IgG Detection by Immunoprecipitation

In one embodiment, the present invention describes a single cell screening method using microbubble well arrays where cells are sorted by what they secrete. Example applications are to sort antigen-specific B cells or cytokine-specific T cells from a heterogeneous leukocyte population. This assay takes advantage of the unique MB well architecture and the small culture volume that enable cells to condition their microenvironment quickly to survive and proliferate. We have shown through experiment and simulation that factors (antibodies, cytokines, growth factors etc.) secreted by cells seeded in MB wells can accumulate to bioactive levels that can affect their function [28]. Using the disclosed assay we can identify the MB well(s) in an array by immunoprecipitation reaction. In order for immunoprecipitation to take place the immunoglobin present must be able to bind a multivalent reporter (antigen, antibody, peptide, nanoparticle etc.) to form large polymerized agglutinates.

### Assay to Enable Immunoprecipitation Detection Using a Multivalent Reporter Constructs

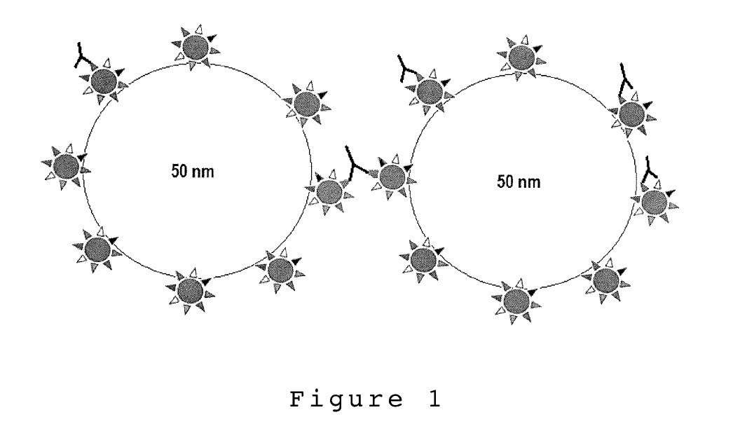

Monoclonal antibodies that bind antigens that display a unique epitope monovalently will not generate a precipitation reaction. One of the most novel approaches to overcome this binding specificity is to conjugate the monovalent antigen to a nanoparticle (NP) to achieve multivalent display. As is understood by those skilled in the art many groups have utilized antigen conjugated NPs as a vaccine delivery device for increased sensitization [34, 35]. While different in its ultimate objective, the conjugation of antigen to NPs for vaccine therapy works on the same principle; the desire to display the antigen epitope multiple times to increase presentation to the mAb for polymerization. On embodiment of this approach is to conjugate antigen to fluorescently activated polystyrene nanoparticles as shown in the schematic. As seen in FIG. 1, antigen multivalency is achieved by displaying the epitope multiple times on the nanoparticle facilitating the cross linking and polymerization between the antigen specific Ig molecules needed to precipitate out a fluorescent signal. Another embodiment of this approach is to conjugate fluorescently tagged antigen to nanoparticles.

### Cell Secreted Product Detection Using Affinity Capture Coating

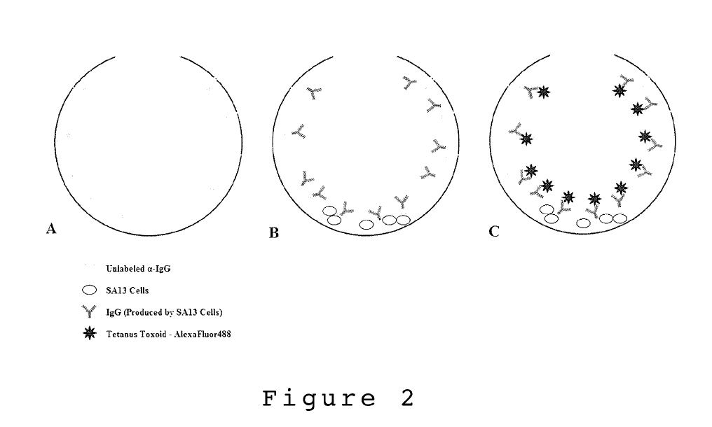

In one embodiment, the present invention includes a single cell screening method using microbubble well arrays where cells are sorted by their secreted products that are bound on the microbubble well surface using affinity capture coatings (FIG. 2). To minimize the cost and time associated with the synthesis of multivalent nanoparticle constructs those skilled in the art will recognize the use of a capture coating to accumulate and concentrate secretions products along the inner wall of the MB will facilitate detection. Cells are cultured for a period of time, the secreted products are captured on the wall which at some point in time is detected using a labeled reporter. Using a fluorescent reporter will result in a more uniform ring signal rather than punctate signal.

### Antigen Specific Detection Using Affinity Capture Coating

In one embodiment, the present invention includes a single cell screening method using microbubble well arrays where cells are sorted by their secreted products that are bound on the microbubble well surface using affinity capture coatings and antigen specific reporter. Detection of Ig and antigen specific Ig can be achieved simultaneously but adding the appropriate reported into the cell culture media on day 0 for the full time the cells remain in culture or it can be added to the cell culture some many days post initiation of the array culture for 2 or 24 hours or many more hours to watch signal develop.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Cells and Cell Lines

Three cell lines were selected for demonstrating the diffusion assay proof-of-concept that relies on the unique feature of the MB well architecture to accumulate cell secreted factors. All cell lines were cultured at 37° C. and 5% CO₂ in RPMI 1640 (Gibco A10491-01, Invitrogen Corp., USA) supplemented with 15% heat inactivated fetal bovine serum (Gibco 10082-147, Invitrogen Corp., USA) and 1% penicillin streptomycin (Gibco 15140-122, Invitrogen Corp., USA). The SA-13 cell line (ATCC HB-8501, Cetus Corp., USA) used secretes IgG with specificity to tetanus toxoid. To validate the growth, proliferation and specific detection of IgG we also used the ARH-77 cell line (ATCC CRL-1621, BD Drewinko) and the CCL-119 T cell line (ATCC CCL-119, GE Foley). ARH-77 cells secrete Ab but with no specificity to tetanus toxoid. The CCL-110 cells do not secrete Ab. A summary of the cell lines with the Ab secretion frequency determined by ELISpot is given in Table 1.

TABLE 1
Cell Line Summary - Cells used for proof-of-concept assays with their
IgG specific secretion percentages as determined by ELISpot.

| | | Antibody | | Secretion |
|---|---|---|---|---|
| Cell Line | Cell Type | Secretion (Y/N) | Specificity | % |
| SA13 | Hybridoma | Y | Tetanus | 25 ± 6% |
| | | | Toxoid | |
| ARH77 | B lymphoblast | Y | Unknown | 7 ± 4% |
| CCL119 | T lymphoblast | N | N/A | N/A |
| PRIMARY | B cell | Y | Variable | 2-4% |

Additionally, primary B cells were used for realistic validation of the MB well array diffusion assay. With Institutional Review Board approval and healthy donor informed consent, peripheral blood mononuclear cells (PBMCs) were isolated using BD CPT (BD Biosciences) tubes. B cells were enriched by negative magnetic selection using the B cell Isolation Kit II (Miltenyi Biotec, San Diego, Calif.) according to manufacturer's instructions. B cells were cultured for three days at 1.5×10⁵ cells per ml in RPMI 1640 media containing 10% FBS and 10 ng/ml IL-2 (Peprotech, Rocky Hill, N.J.), 2.5 μg/ml CpG2006 (IDT, Coralville, Iowa), 2.5 μg/ml R848 (Invivogen, San Diego, Calif.), and 1:100,000 Staphylococcus aureus Cowan (SAC) (EMD Millipore, Darmstadt, Germany). After three days, B cells were collected, washed twice, and re-suspended in media above without SAC and seeded into MB well arrays.

### Example 1

### Detection of IgG by Immunoprecipitation

In one embodiment, the present invention describes a single cell screening method using microbubble well arrays where cells are by what they secrete using immunoprecipitation detection. For immunoprecipitation to take place the immunoglobin present must be able to bind a multivalent reporter (antigen or antibody) to form large polymerized agglutinates. Once a sufficient size threshold is reached the agglutinates settle forming a precipitate [33]. For example, SA13 hybridoma cells were used to demonstrate the accumulation of IgG secretions in the MB well using immuno-precipitation detection. Following the seeding of SA13 cells into the MB array, a rabbit α-human IgG-FITC reporter (Rockland antibodies and assays #209-4202, USA) was added to the culture media. The chips were incubated with the reporter over 3-4 days and imaged daily using an inverted fluorescent microscope (Olympus IX70 with Qlmaging Retiga EXi camera). Images were taken in both bright-field and fluorescence (FITC filter 450-480 nm) for 20 ms and 518 ms respectively. Images were then artificially colored and processed using the enhance contrast function in ImageJ (NIH).

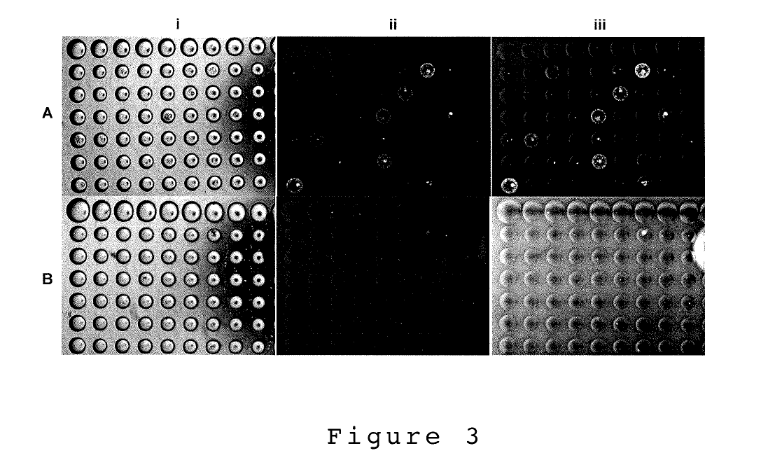

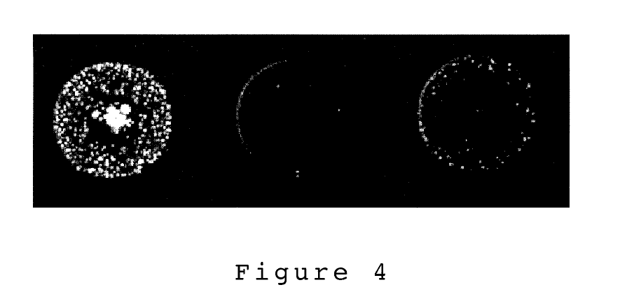

For example, SA13 hybridoma cells were used to demonstrate the accumulation of IgG secretions in the MB well using immuno-precipitation detection (FIGS. 3 and 4). Following the seeding of SA13 cells into the MB array, a rabbit α-human IgG-FITC reporter (Rockland antibodies and assays #209-4202, USA) was added to the culture media. The chips were incubated with the reporter over 3-4 days and imaged daily using an inverted fluorescent microscope (Olympus IX70 with Qlmaging Retiga EXi camera). Images were taken in both bright-field and fluorescence (FITC filter 450-480 nm) for 20 ms and 518 ms respectively. Images were then artificially colored and processed using the enhance contrast function in ImageJ (NIH). FIG. 3 shows an example fluorescent image of a region of a MB array in which a Hybridoma cell line (SA13) was seeded and culture for 6 days in media containing FITC-anti-IgG antibody (1 ug/ml). Based on Elispot measurements, 20% of SA13 cells secrete antibody against tetanus toxoid. It is evident that wells containing these cells are bright and evidence of speckle from immunoprecipitation is clear even without image enhancement. A magnified view of a high positive, a presumed negative, and low positive wells are shown in FIG. 4.

### Example 2

### Tetanus Toxoid Antigen Specific Immunoglobin Detection by Immunoprecipitation Using a Nanoparticle Multivalent Reporter Construct

Nanoparticle Synthesis

Clostridium tetani Tetanus Toxoid (Pierce Antibodies HYB 278-17-02, Thermo scientific, USA) was conjugated to carboxyl terminated polystyrene nanoparticles with an average size of 50 nm (Polylink Polysciences, Inc., USA) through covalent coupling via EDC. The conjugation was done by the manufactures specifications with some procedural alterations. Due to cost associated with tetanus toxoid, 3 ug of tetanus toxoid was added to the NP suspension, this was approximately 100× less antigen than the procedure called for to obtain complete PS-NP antigen saturation. Additionally, the toxoid-NP incubation was done over the course of 2 hours with light mixing. This was in contrast to the manufactures recommendation of a 1 hour incubation period. This was done to increase likely hood of NP-toxoid conjugation due to the relatively low levels of toxoid added to the suspension. Following conjugation the PS-NPs were stored at 4° C. until needed.

Dynamic Light Scattering Instrumentation

To confirm the conjugation of tetanus toxoid to the nanoparticles surface dynamic light scattering measurements (DLS) were conducted to measure both hydrodynamic diameter changes as well as zeta potential shifts before and after conjugation. Both functionalized and unfunctionalized (Stock) nanoparticles were diluted to a concentration of 75 ug/ml in deionized water with a pH of 6.8-7. Using a Malvern Zetasizer Nano ZS (Malvern Instruments Inc., USA) size and zeta potential measurements were collected. From Table 2 it can be seen that following conjugation of the toxoid to the PS-NPs the sizes of the PS-NPs increased dramatically and the zeta potential shifted by approximately 30%.

TABLE 2
Nanoparticle Characterization - Following conjugation of the toxoid the
PS-NP experienced a shift in zeta potential as well as an increased in size.

| Type | Size (d · nm) | Zeta (mV) |
|---|---|---|
| Un-conjugated | 54.8 | −43 |
| Conjugated | 325 | −30.5 |

Before conjugation with the toxoid the PS-NPs remain highly stabile, shown both by the DLS measurements above as well as from visual inspection after 72 hour storage. The dramatic increase in PS-NP size as well as the settling of the NPs after extended storage periods indicates the particles may be unstable in solution following the conjugation process, resulting in them settling or precipitating out of solution.

Nanoparticle Detection Protocol

Chips were prepared, sanitized and seeded as mentioned above. For the initial experiment only SA13 cells were used. On day 4 of incubation the PS-NP conjugated to the tetanus toxoid were ultrasonicated and then added to the media at a concentration of 1 ug/mL. Following an 18-24 hour incubation with the PS-NPs the cells were imaged using the protocol described in section 2.2.5. Future PS-NP tests were done using the same protocol as previously mentioned with the addition of a second MB array which was seeded with ARH-77 cells as a negative control sample. For the comparison study a wash protocol was developed to minimize nonspecific interactions. Both chips were stored upside down in fresh RPMI media for 2.5 hours following the 24 hour incubation with the PS-NP.

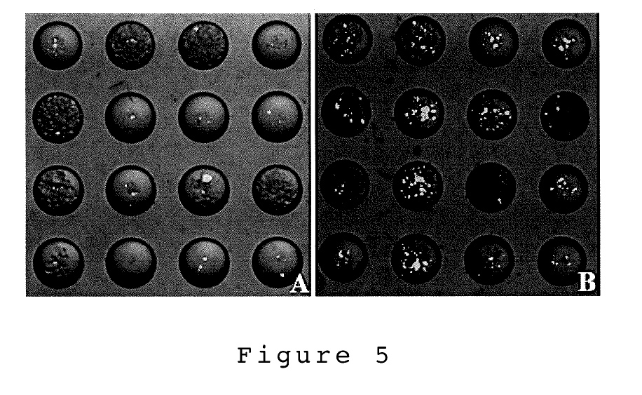

Despite issues associated with stability of the PS-NPs over extended periods of time the PS-NPs were added to the MB arrays as outlined above. The first experiment with the PS-NP detection system was to compare the functionalized PS-NPs with unfunctionalized PS-NPs to compare specific binding with nonspecific interactions or PS-NP settle. As seen in FIG. 5, results showed dramatic qualitative increases in fluorescent readouts between the functionalized and unfunctionalized PS-NPs when added to a MB array seeded with SA13 cells after 4 days of incubation.

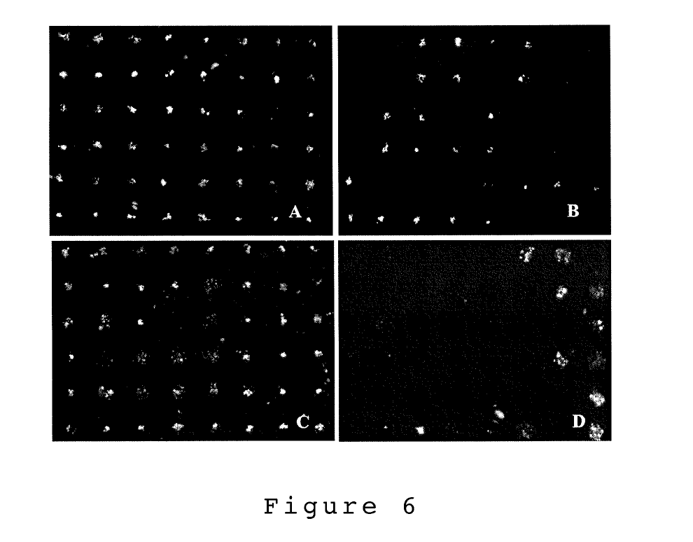

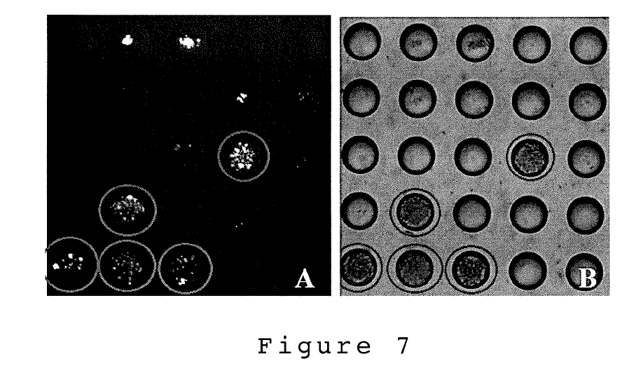

Following the preliminary testing with PS-NP signal enhancement, further tests were conducted to confirm the initial results. ARH-77 and SA13 cells were seeded into two separate MB arrays at 15,000 cells/cm² and tetanus toxoid functionalized PS-NPs were added to the media in the same fashion as the first experiment. While ARH-77 cells produce IgG, the Abs secreted from the ARH-77 line hold no specificity for tetanus toxoid. Therefore, only the SA13 Ab secretion should theoretically bind the tetanus toxoid functionalized PS-NP. FIG. 6 shows the initial results of the ARH-77 and SA13 PS-NP comparison. From FIGS. 6(A&C) it can be seen that both the SA13 line and the ARH-77 line produced high levels of IP. This is inconsistent with the hypothesis due to the mismatch in specificity between the ARH-77 Ab and the tetanus toxoid PS-NP. Due to the instability of the PS-NPs as discussed earlier in this section it was believed that the signal in the ARH-77 sample was due to non-specific binding or settling. To overcome this, a wash protocol was developed to minimize non-specific interactions as outlined in above. Following the wash procedure it becomes clear that most of the fluorescence retained by the ARH-77 sample is punctate or spot fluorescence, while the fluorescence that remains in the SA13 sample is diffuse and mimics the speckle pattern originally seen in Example 1 above. Furthermore, closer inspection reveals that nearly all of the speckle fluorescence present in the SA13 sample corresponds to wells which contain a plethora of cells within them (FIG. 7).

### Example 3

### Cell Secreted IgG Detection Using Affinity Capture

One embodiment, the present invention includes sorting cells by detecting antibody secretions. The general procedure used to prepare microbubble well array chips for affinity capture of secreted antibody products (IgG, IgM, IgE, IgA, IgD and all isotypes) is similar. The procedure used to detect IgG is briefly described. Array chips are sterilized in ethanol and incubated in 1×PBS for 24 hour to allow for the removal of residual ethanol. For the detection of IgG the chips are then transferred into new wells of a 24 well plate and 1 mL of a 0.01 mg/mL goat α-human IgG:PBS mixture is added (Rockland antibodies and assays #209-1102, USA) and left to incubate at room temperature for 90 minutes, allowing for the nonspecific binding of α-IgG to all surfaces. Those skilled in the art know that capture antibody can be oriented using standard biotinylation with streptavidin or Protein G/A protocols. The chips are then transferred to new wells containing a 2% bovine serum albumin (BSA) in 1×PBS for 20 minutes to decrease non-specific interactions. Following the BSA block, the chips are transferred into wells containing fresh RPMI 1640 (Gibco A10491-01, Invitrogen Corp., USA) for 10-15 minutes at 37° C. The cells are then seeded and the rabbit α-human IgG FITC reporter (Rockland antibodies and assays #209-4202, USA) is added to the culture media. Cells are cultured for a period of time 1-10 days until signal develops that appears a fluorescent ring. Those skilled in the art know that various reporter constructs can be used including other fluorescent dyes and chromogens.

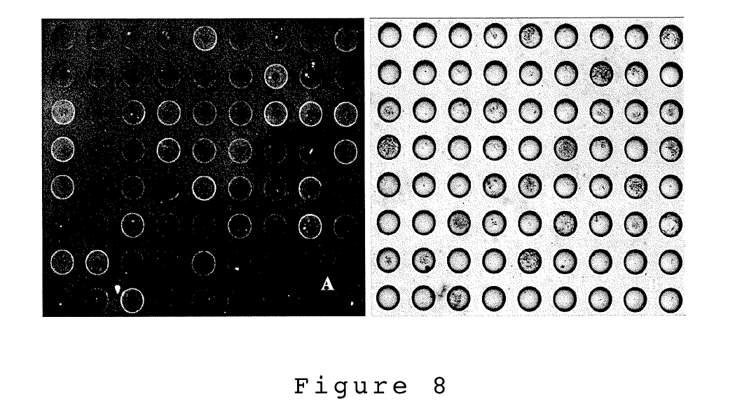

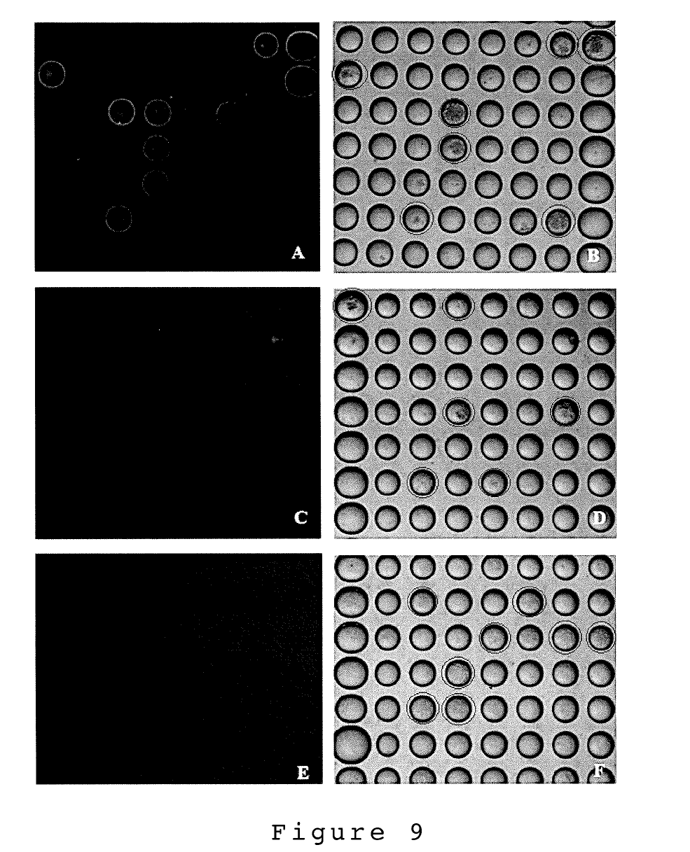

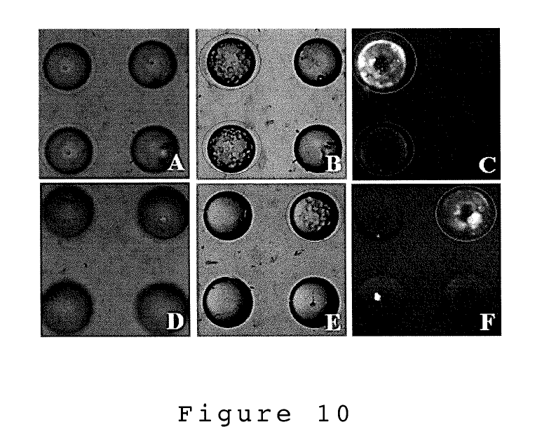

SA13 cells were seeded in a MB well array coated with unlabeled human α-IgG and cultured with AlexaFluor488-tetanus toxoid for 4 days. Portions of an array containing (72 MB wells) imaged in fluorescence and bright-field are shown in FIGS. 8A and 8B, respectively. It can be seen that the coating facilitated the capture and concentration of tetanus specific IgG along the surface of the MB well with positive wells indicated by presence of high contrast rings as opposed to a speckle pattern. To confirm this result, affinity coating controls were run with ARH-77 and CCL-119 cells which do not secrete toxoid specific mAbs. Results showed that only the chips containing SA13 cells produced positive fluorescence rings when cultured with AlexaFluor488-tetanus toxoid (FIG. 9). It is interesting to note that while some of the MB wells in FIG. 8B show cell presence more apparently than others (white arrows) the magnitude of the ring intensity does not correlate with the number of cells in the well. This is not unexpected as Elispot data indicated that just ˜25% of the SA13 cells secrete tetanus toxoid specific IgG. A further example of this is illustrated in FIG. 10A-F. MB wells that started with single cells (FIGS. 10A, 10D) and clonally proliferated to a comparable number of cells after 4 days (FIGS. 10B, 10E) produced markedly dissimilar fluorescence signal (FIG. 10C, 10F). MB wells which start with single cells that have low secretion rates likely give rise to clonal cell populations that also will have characteristically low secretion rates and thus produce minimal fluorescence regardless of cell number per well. Antibody secretion rates can vary over a wide range; 10 to 10⁵ ftg/cell/hr [36]. If antibody is produced by a single cell at ˜0.5 pg/hr its concentration in a 0.9 nL MB well could rise to ˜13 μg/mL in 24 hr or ˜53 μg/mL over 4 days.

### Example 4

### Tetanus Toxoid Antigen Specific Immunoglobin Detection Using Affinity Capture Coating

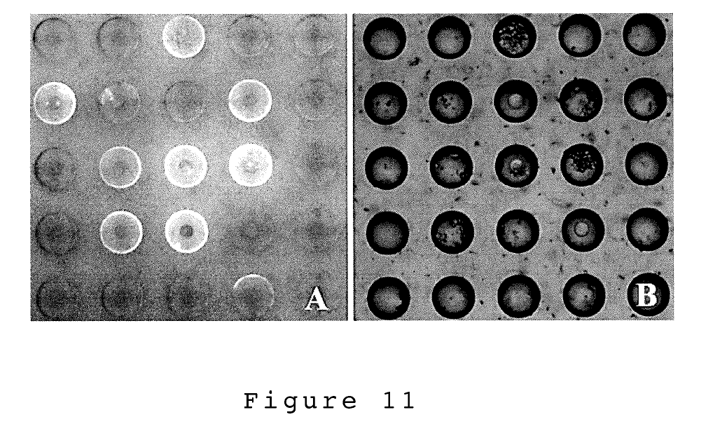

The chip preparation protocol was carried out the in same way as described in Example 3 for detecting IgG however the reporter used to detect the tetanus toxoid specific mAb. In this example, to detect tetanus toxoid specific mAb produced by SA13 cells the antigen Clostridium tetani Tetanus Toxoid was conjugated to AlexaFluor488. Briefly, 50 μg Tetanus toxoid (4582231, Calbiochem) was dialyzed into PBS and was then adjusted to pH˜8.3 with 1M sodium bicarbonate. The conjugation reaction between Tetanus toxoid and AlexaFluor488 dye through tetrafluorophenyl ester was performed at room temperature for 1 hour using the AlexaFluor488 labeling kit (#A20181, Molecular Probes). Then conjugated protein was separated from free dye by spinning through purification resin column. Then the volume was adjusted to 100 μl (0.5 μg/ml) in PBS. In this example assay the immunoglobin secreted by cells is captured by the anti-immunoglobin coating on the MB well surface. Over time the fluorescently labeled antigen—in this example AlexaFluor488-tetanus toxoid—diffused into and is captured by the anti-toxoid antibody (FIG. 11) producing high contrast ring signal in positive MB wells.

### Example 5

### Assay to Sort Human Peripheral B Cells that Secrete Antibody Against HIV Envelope gp140 Antigen

In one embodiment, the present invention includes a single cell screening method using microbubble well arrays where antigen specific B cells isolated from human peripheral blood are sorted using affinity capture coatings and antigen specific detection. An outline of how the assay works for sorting human B cells that secrete antibody against HIV envelope gp140 antigen is as follows:
Step 1: A MB well array chip is prepared for cell seeding—process steps include ethanol sterilization of the chip, gas plasma treatment to render chip backside hydrophilic, BSA treatment to block nonspecific binding on chip topside, vacuum treatment to prime MB with PBS, reagent exchange to prefill MB wells with cell culture media.
Step 2: B cells from a patient sample are obtained—this is a heterogeneous population of B cells that can make antibodies to a myriad of different kinds of pathogens. Some patients with HIV may make antibodies against gp140 or other viral epitopes that we could assay for instead or simultaneously.
Step 3: B cell sample is seeded in the MB array under limiting dilution conditions. We have developed and extensively characterized MB cell seeding protocols and determined the importance of cell solution concentration and the size of the MB opening as key parameters that determine cell distribution statistics in wells [30]. Seeding at 5000 cells/cm² on chips containing MB wells with circular openings 60 microns in diameter typically yields ˜76% empty MB wells and ˜20% of the MB wells contain 1 cell. The remaining 4% of MB wells contain 2 or more cells. Low cell seeding enables proliferation of clonal pure cultures. Higher cell seeding densities can be used. This may favor cell survival but cultures will not be clonal and recovery of cells will be mixed.
Step 4: Arrays are cultured in activating stain media. B cells are cultured in media that contain additives to stimulate them to produce and secrete antibody. These stimulatory cocktails can vary widely but typically they contain IL2. The stain media also contains a fluorescently labeled reporter. The reporter can be the antigen which in this example is GP-140 protein. The fluorescent label can be any standard dye (eg. FITC, Alexaflour 488) or quantum dot. It is also possible to use a fluorescently tagged antibody such as anti-IgG. The latter will detect MB wells containing cells that secrete IgG as well as cells with surface bound IgG.
Step 5: Arrays are analyzed over time. The cultures are monitored over time (1 to 7 days or longer if needed) using an inverted microscope to identify fluorescent wells. Wells that contain B cells that secrete the antibody will accumulate the reporter antigen (or reporter antibody). An immuno-precipitation reaction may occur which can be identified as speckle or ring signal can be detected using affinity capture coatings as described in Examples 1, 2 and 3.

### Example 6

### IgG Detection Limit Study

Preliminary studies were conducted in to investigate the IgG detection limit in the MB well array system. MB well chips were fixed in a 48 well plate, sterilized with 50% ethanol and incubated in an oven at 60° C. overnight. The chip surfaces were blocked by placing 600 μL of a 2% BSA solution on unprimed chips (air in the MB wells) at room temp for 30 minutes. The chips were rinsed 4 times with 1×PBS. To coat inside of the the MB wells with capture agent 600 μL of a 0.01 μg/μL solution of unconjugated anti-Human IgG in PBS was added to the wells of a 48 well plate containing chips. The plate was placed in a desk top vacuum for ˜15 minutes until all MB wells were primed with the solution. The plate was then incubated at room temperature on rocker table at low speed for 1.5 hours. After, the chips were rinsed 4 times with 1×PBS and the last rinse was allowed to incubate for ˜15 minutes. The chips were then treated with 600 μL of 2% BSA for 10 minutes at 37° C. and rinsed 4 times with 1×PBS and again the last rinse was allowed to incubate for ˜15 minutes. Next the chips were exposed to 600 μL of either 1 μg/mL, 0.1 μg/mL, 0.01 μg/mL, or 0.001 μg/mL Human IgG in 1×PBS and allowed to incubate for 1.5 hrs at room temp on rocker table at low speed, and then at 37 C.° for another final 30 minutes. The chips were rinsed 4 times with PBS and the last rinse was allowed to incubate for ˜20 minutes. Following this the chips were exposed to 0.002 μg/μL TexasRed conjugated anti-Human IgG in 1×PBS and allowed to incubate in at 37° C. for ˜18 hours. The chips were then rinsed 4 times with PBS and fresh PBS was added to each well prior to imaging.

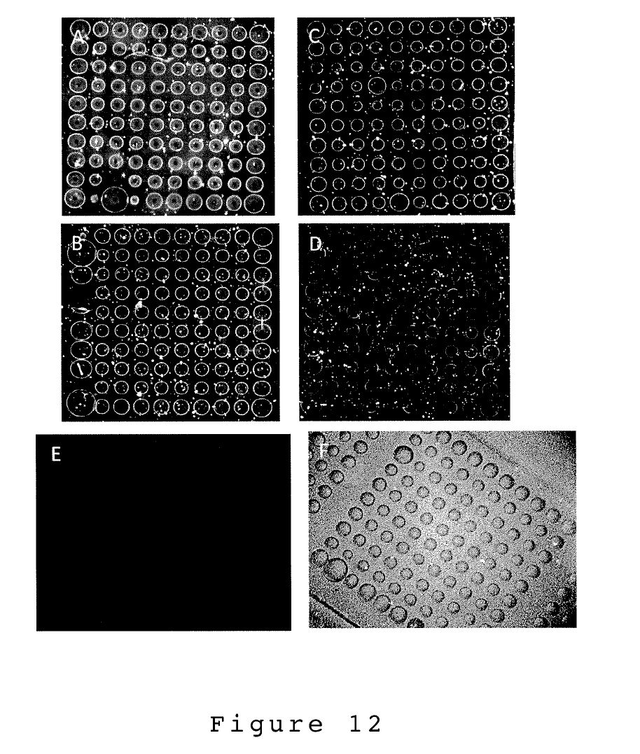

Signal detection limit results (FIG. 12) indicate that an IgG concentration of 1 ng/ml can easily be detected with the current imaging system. However, experimental modifications such as reducing the MB well size, increasing the camera integration time, and use of nonphotobleaching fluorescent reporters could easily push the detection limit to the pg/ml level or lower.

### Example 7

### Culturing of Primary Human B Cells and Detection of Secreted IgG

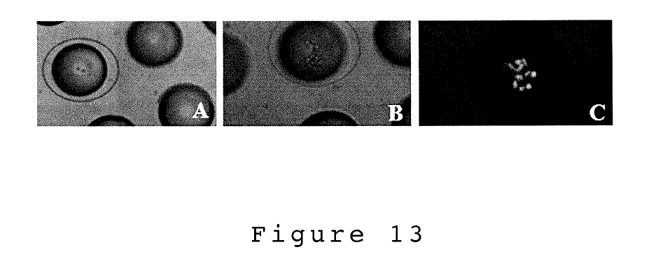

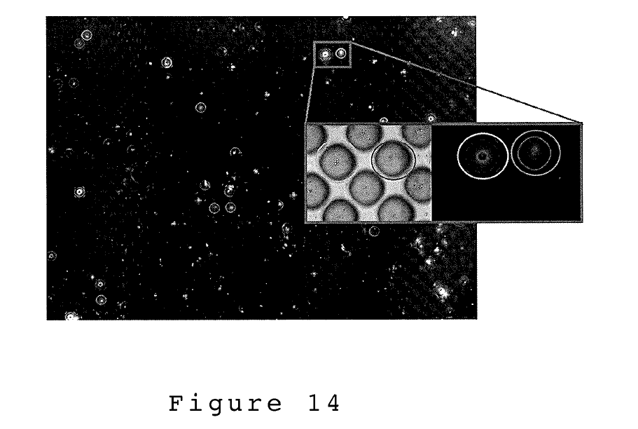

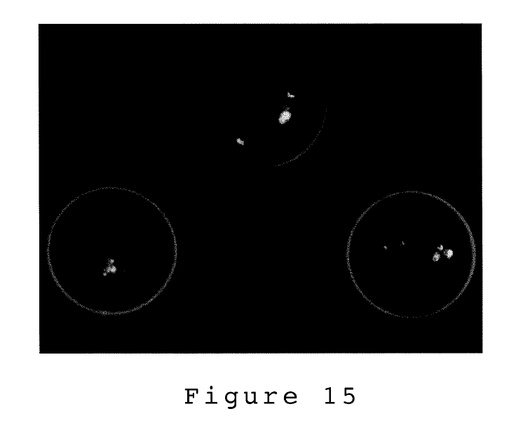

B cells negatively selected from human PBMCs and cultured in MB well arrays showing proliferation occurs FIG. 13. Detection of secreted IgG from human B cells using the affinity capture method is shown in FIG. 14. A zoomed in image showing live human B cells secreting IgG is evident in FIG. 15.

### Example 8

### Recovering Cells from MB Wells

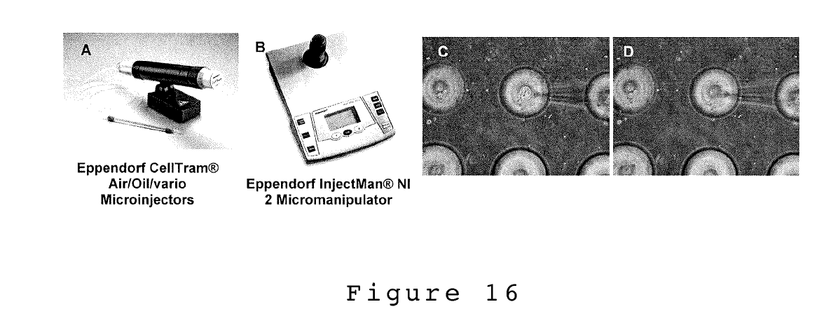

Cell(s) can be recovered from the MB well(s) using commercial micromanipulation tools. After identifying specific MB wells containing cells of interest the cells can be recovered using commercial Eppendorf CellTram and InjectMan NI 2 Micromanipulator technology (FIG. 16).

### Example 9

### PCR Amplification of Ig Genes from B Cells Recovered from MB Wells

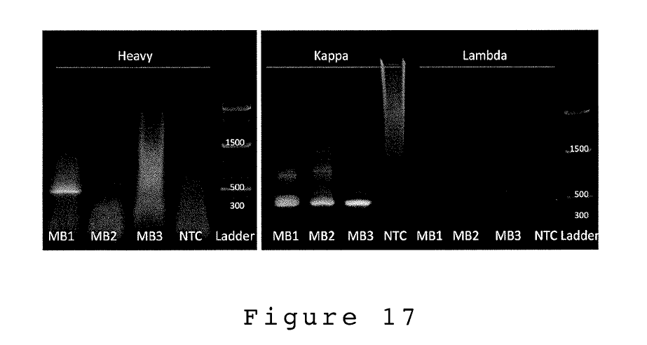

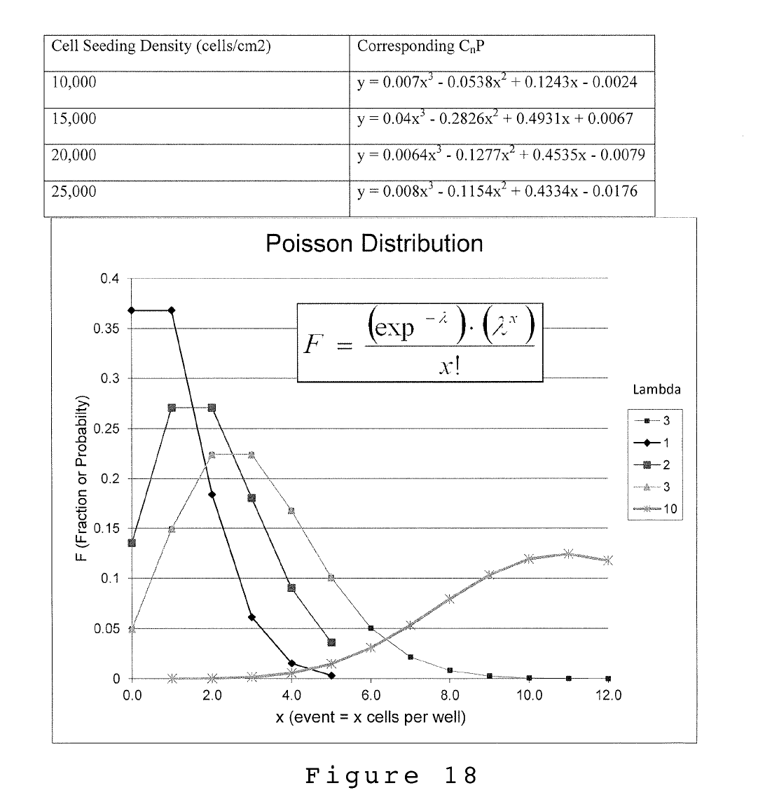

The immunoglobin genes can be cloned and recombinant monoclonal antibodies can be developed, and inform vaccine design (FIG. 17). RT-PCR was done on SA13 hybridoma cells retrieved from MB wells to demonstrate amplification of the heavy, kappa and lambda regions of the immunoglobin genes. MB wells were seeded with single SA 13 cells and cultured for 2 to 3 days to proliferate to 5-8 cells. Manual recovery of cells from MB wells was achieved using commercial Eppendorf CellTram and InjectMan® NI 2 Micromanipulator technology. Cells were transferred into a standard 250 μL PCR tube. RT-PCR techniques from Richardson et al. [37] were followed with the following exceptions. Cells were placed in tubes containing 10 μl/well 0.5×PBS containing 10 mM DTT (Invitrogen, Carlsbad, Calif.), and 8 U RNAsin (Promega, Madison, Wis.). cDNA was synthesized in a total volume of 20 μl/tube using the qScript cDNA Synthesis Kit (Quanta Biosciences). Primer sequences for the immunoglobin lambda, kappa, and heavy chain genes are given in FIG. 18.

### Example 10

### Predicting the Frequency of Antibody Secreting Cells

It should be noted that the MB well array system can be used to approximate the frequency of ASC, expressed as a percentage of cells in the heterogeneous sample, which is typically measured using ELISpot. With knowledge of the initial cell seeding distribution (# of MB wells seeded with 1, 2, 3, or 4 cells), the % of ASC can be estimated by simply counting the number of fluorescent rings using Equation 1.

$$\% IgG = \frac{{R}_{*}}{{\sum }_{n}^{4} ( {C}_{n} P * {MB}_{T} )} \qquad ( Equation 1 )$$

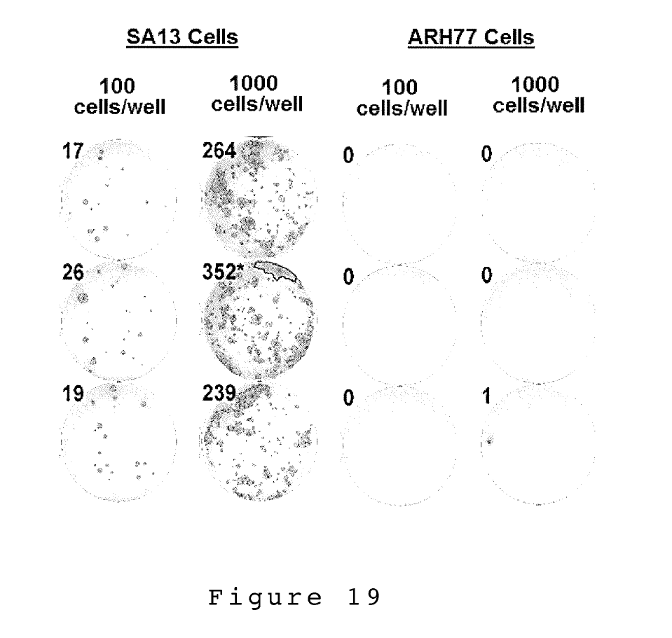

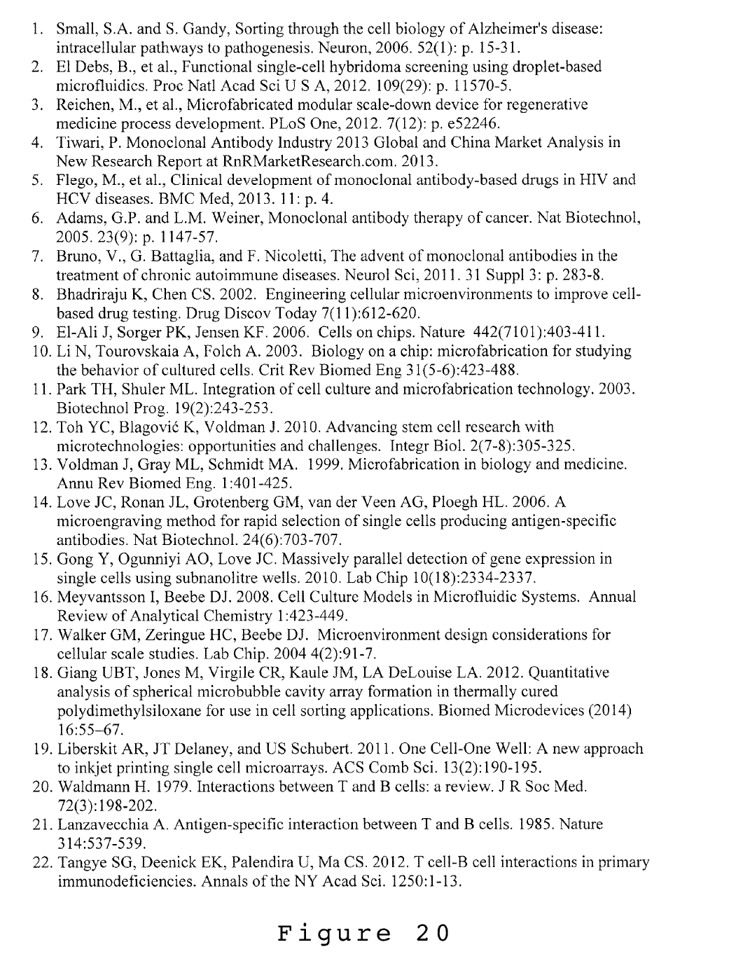

In this equation R* is the number of wells with immunoprecipitates or fluorescent rings within the field of view while MB_{T} is the total number of MB wells within that same window of view. The CₙP term is unique to the cell seeding concentration which determines the percentage of MB wells with n=0, 1, 2, 3 or 4 cells (FIG. 19). Using this equation and the fluorescent image of the primary B cell array (FIG. 14) we estimated that the % percentage of IgG secreting was 2.7±1%, versus the actual value obtained from ELISpot which was found to be 6.4±4.2% (FIG. 20). The accuracy of this prediction relies on the ability to reproducibly seed arrays with a consistent distribution. Studies suggest that the MB well seeding statistics (CₙP) across various cell lines is consistent for a given cell seeding concentration and MB well opening. Hence, MB well array technology has the potential to glean data on the frequency of ASC and the antibody secretion rate in a single assay which enables this technology to compete directly with more conventional techniques such as ELISA and ELISpot, all while doing so more efficiently and with higher throughput.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method using a microfabricated device for cell culture, sorting and analysis in situ or ex situ, where the microfabricated device comprises one or more curvilinear microbubble well cavities embedded in preferably a non-glass substrate, where the opening into the cavity is smaller in diameter than the diameter of the cavity is at its largest extent so as to create a microenvironmental niche into which one or more cells can be seeded and cultured for a period of time of hours to days to weeks so that their secreted product(s) can accumulate to promote cell survival and/or proliferation

2. A method using a microfabricated device in claim 1 with cavity openings of 20-200 microns in diameter.

3. A method using a microfabricated device in claim 1 with cavity openings of 40 to 60 microns in diameter.

4. A method using a microfabricated device in claim 1 with circular, triangular, rectangular, or square cavity openings.

5. A method using a microfabricated device in claim 1 with a maximum cavity diameter that is larger than the cavity opening.

6. A method using a microfabricated device in claim 1 with a maximum cavity diameter that is about 2 to 4 times larger than the cavity opening.

7. A method using a microfabricated device in claim 1 comprised of one or more cavities in an array.

8. A method using a microfabricated device in claim 1 with an array of cavities from 2 to 1 million or more.

9. A method using a microfabricated device in claim 1 comprising a substrate material with an elastic modulus similar to in vivo tissue microenvironment.

10. A method using a microfabricated device in claim 1 comprising a substrate material with an elastic modulus similar to in vivo soft tissue microenvironment.

11. A method using a microfabricated device in claim 1 where the substrate material has an elastic modulus in the range of 100 to 1000 KPa.

12. A method using a microfabricated device in claim 1 that is comprised of a polymer substrate material with an elastic modulus in the range of 100 to 1000 KPa.

13. A method using a microfabricated device in claim 1 where the substrate material is a clear polymer material with elastic modulus in the range of 100 to 1000 KPa such as polydimethylsiloxane (PDMS).

14. A method using a microfabricated device in claim 1 into which cells are seeded.

15. A method using a microfabricated device in claim 14 where the cells are selected from the group consisting of mouse hybridoma cells, CHO cells, or B cells derived from human or animal peripheral blood or lymphoid organs.

16. A method using a microfabricated device in claim 1 into which the number of cells seeded per cavity is controlled.

17. The method of claim 16, where the number of cells seeded per cavity follows a statistical population.

18. The method of claim 17, where the number of cells seeded per cavity follows a statistical population defined by Poisson distribution.

19. A method using a microfabricated device in claim 1 into which the number of cells seeded per cavity follows a statistical distribution with preferred seeding of ˜37% of the cavities have 0 cells, ˜37% have 1 cell, ˜18% have 2 cells and ˜8% have 3 cells.

20. A method using a microfabricated device in claim 6 that provides a microenvironmental niche for a single cell or multiple cells seeded into a cavity to readily condition with autocrine or paracrine secreted factors to support cell survival and clonal or colony proliferation.

21. A method using a microfabricated device in claim 14 further configured to permit visual inspection of cells in cavities after seeding and over time to quantify cell proliferation so as to distinguish cells that die from cells that undergo rapid proliferation.

22. A method using a microfabricated device in claim 14 into which the seeded cells are cultured for a period of time ranging from hours to several days in media that contains supplements and reporters.

23. A method using a microfabricated device in claim 22 in which the reporter is a fluorescently or chromogenic tagged antigen, peptide, cytokine, antibody or other protein or nanoparticle that will bind to cell secreted factors.

24. A method using a microfabricated device in claim 14 in which the reporter binds to cell secreted factors causing a precipitation reaction.

25. A method using a microfabricated device in claim 1 in which the cavities are coated with a functional biomolecule and the device-surface is coated separately with a cell and/or protein blocking reagent.

26. A method using a microfabricated device in claim 25 where an unprimed chip having air in the microbubble wells is exposed to a high surface tension liquid containing a coating protein.

27. A method using a microfabricated device in claim 26 where the liquid has a surface tension >40 dyne-cm, and preferably 70 dyne-cm.

28. A method using a microfabricated device in claim 26 where the coating protein is a blocking agent such as bovine serum albumin, casein, polyethyleneglycol (PEG) or other blocking agents know in the field.

29. A method using a microfabricated device in claim 26 where the coating protein is allowed to react with the chip surface for a period of time from 1 to 24 hours at room temperature (RT) or at 4 C, preferably 2 hrs at RT.

30. A method using a microfabricated device in claim 26 where the coating protein is allowed to react with the chip surface for a period of time from 1 to 24 hours at room temperature (RT) or at 4 C, preferably 2 hrs at RT.

31. A method using a microfabricated device in claim 26 where the coating protein is washed off and replace with a buffer.

32. A method using a microfabricated device in claim 31 where the chip immersed in buffer is placed in a vacuum to draw the buffer into the microbubble wells to attain a primed array.

33. A method using a microfabricated device in claim 32 in which the primed array is placed in a second solution containing a bioactive molecule that will coat the inside of the microbubble cavity.

34. A method using a microfabricated device in claim 33 where the bioactive molecule is allowed to react with the microbubble well surface for a period of time from 1 to 24 hours at room temperature (RT) or at 4 C, preferably 2 hrs at RT.

35. A method using a microfabricated device in claim 33 in which the bioactive molecule can affect cell function such as an extracellular matrix protein or capture cells or cell secreted products on the cavity surface.

36. A method using a microfabricated device in claim 25 in which the cell secreted products that are captured on the cavity surface are detected with a fluorescently or chromogenic tagged antigen, peptide, cytokine, antibody or other protein or nanoparticle reporter.

37. A method using a microfabricated device in claim 1 or 25 in which the cell secreted products that precipitate or are captured on the cavity surface are detected with a reporter for which the signal strength in individual cavities is monitored over time to identify cavities containing cells that secrete product early and at a fast rate.

38. A method using a microfabricated device in claim 1 or 25 into which two or more distinct cells types are seeded into the microcavity niche to observe functional readouts.

39. A method using a microfabricated device in claim 14 or 25 into which cells are seeded in micro-niche cavities and cultured in time (hours to days to weeks) to observe clonal proliferation and morphology.
